(19) United States
(12) Patent Application Publication
(10) Pub. No.: US 2025/0249239 A1
(43) Pub. Date: Aug. 7, 2025

(54) Vector Potential Coil Device That Applies Electrical Stimulation To Skin

(71) Applicants: SUMIDA CORPORATION, Tokyo (JP); NATIONAL UNIVERSITY CORPORATION, IWATE UNIVERSITY, Iwate (JP)

(72) Inventors: Kenji Terao, Miyagi (JP); Masaki Saito, Miyagi (JP); Masahiro Daibo, Morioka (JP)

(21) Appl. No.: 19/187,283

(22) Filed: Apr. 23, 2025

Related U.S. Application Data
(63) Continuation of application No. PCT/JP2023/026194, filed on Jul. 18, 2023.

(30) Foreign Application Priority Data
Nov. 22, 2022 (JP) 2022-186155

(51) Int. Cl. A61N 1/32

(52) U.S. Cl. CPC A61N 1/328

(57) ABSTRACT

A support part has a housing space that accommodates skin of a living body. A vector potential coil in a vector potential coil device is arranged at at least one of the housing space and an outside of the support part. A power supply section conducts an alternating current through the vector potential coil, generates a vector potential corresponding to the alternating current in the housing space, and provides an electrical stimulation to the skin by applying an electrical field generated based on the vector potential to the skin of the living body.

### CROSS REFERENCE TO RELATED APPLICATIONS

This application is a continuation of PCT Application No. PCT/JP2023/026194, filed on Jul. 18, 2023, which claims priority to Japanese Patent Application No. 2022-186155, filed on Nov. 22, 2022. The contents of both of the above applications are expressly incorporated herein by reference in their entirety.

### BACKGROUND

### Technical Field

The present invention relates to a vector potential coil device that applies electrical stimulation to skin and an application method of the same.

### Related Art

In recent years, there have been great advances in research into dermatology and cosmetic science. The number of cases, in which conventional drug therapies such as methods of external applications, internal medications, and so on are not sufficiently effective, is also increasing. As a result, various proposals have been made, including phototherapy such as lasers and photofacials, or methods of applying electrical and/or magnetic stimulations.

For instance, for a cosmetic treatment, a cosmetic device has been proposed (for instance, refer to International Patent Publication Number WO2022/124140). The cosmetic device includes a skin electrode that comes in contact with a user's skin to apply an electrical stimulation thereto, a stimulus generating part that generates the electrical stimulation, and a conductive part that conducts between the skin electrode and the stimulus generating part and transmits the electrical stimulation generated by the stimulus generating part to the skin electrode. Further, there has also been proposed a beauty machine (for instance, refer to Japanese Patent Publication Number 2007-236699). In regard to the beauty machine, contact surfaces of two rollers that come in contact with such as a facial skin are each shaped to a predetermined length and arranged in substantially parallel to each other with a predetermined distance between them, so that the area being sandwiched between the two contact surfaces becomes a relatively large treatment area.

On the other hand, a vector potential generation device has been developed (for instance, refer to International Patent Publication Number WO2015/099147). The vector potential generation device generates a vector potential by conducting a current through a vector potential coil that is formed by circulating a solenoid coil. Furthermore, a vector potential detection device has also been developed (for instance, refer to Japanese Patent Number 6950925). The vector potential detection device detects a vector potential by utilizing a state in which a voltage is induced by a temporal change (varying in time) of the vector potential.

In the above-mentioned electrical and/or magnetic stimulation method, it is necessary to apply a current while manually or mechanically moving a roller-type weak current generator along the skin. Therefore, there is a concern about applying a stress to or injuring the skin during the treatment or operation and at the same time, it is inconvenient that it requires human power or mechanical power. Therefore, a treatment device, which can minimize a damage to such as skin and at the same time, can save a labor while having the effect of a weak current, is expected.

### SUMMARY

The present invention has been made in view of the above and has an object that is to obtain a device, which treats (cures) skin diseases and skin wounds and performs cosmetic treatments via an electric field stimulation while applying an appropriate electrical and/or magnetic stimulation to the skin, and at the same time, suppressing the occurrence of problems caused by contacting with the skin.

A vector potential coil device according to the present invention includes a support part having a housing (or accommodation) space that houses skin of a living body, a vector potential coil that is placed at at least one of the housing space of the support part and an outside thereof, and a power supply section that conducts an alternating current through the vector potential coil, generates a vector potential corresponding to the alternating current in the housing space, and applies an electric field generated based on the vector potential to the skin of the living body, thereby providing an electrical stimulations to the skin.

### Effects of the Invention

According to the present invention, for instance, it is possible that a vector potential coil device can be placed at a position that is away from skin of a living body by several centimeters to ten and several centimeters and an electric field and/or a current can be applied from that position. As a result, a degree of a stress and an injury to the skin can be reduced, and at the same time, a labor can be saved. Therefore, it is possible to obtain a labor-saving vector potential coil device which treats (cures) a skin disease and a wound and performs a cosmetic treatment via an electrical stimulation while suppressing the occurrence of problems caused by an electrode for the electrical stimulation to the skin of the living body.

### BRIEF DESCRIPTION OF THE DRAWINGS

FIG. 1 is a block diagram that shows a configuration of a vector potential coil device 1 according to an embodiment of the present invention.

FIG. 2 is a diagram that shows an example of a support part 10 and a vector potential coil device 1 according to a first embodiment.

FIG. 3 is a diagram that shows an example of a vector potential coil device 1 according to a second embodiment.

FIG. 4 is a diagram that shows an example of a vector potential coil device 1 (a part) according to a third embodiment.

FIG. 5 is a diagram that shows an example of a support part 10 and a vector potential coil device 1 according to a fourth embodiment.

FIG. 6 is a side view that shows an example of the vector potential coil device 1 according to the fourth embodiment.

FIG. 7 is a top view that shows an example of a vector potential coil device 1 according to a fifth embodiment.

FIG. 8 is a top view that shows an example of a vector potential coil device 1 according to a sixth embodiment.

FIG. 9 is a top view that shows an example of a vector potential coil device 1 according to a seventh embodiment.

FIG. 10 is a top view that shows an example of a vector potential coil device 1 according to an eighth embodiment.

FIG. 11 is a top view that shows an example of a vector potential coil device 1 according to a ninth embodiment.

FIG. 12 is a side view that shows an example of a vector potential coil device 1 according to a tenth embodiment.

FIG. 13 is a top view that shows an example of the vector potential coil device 1 according to the tenth embodiment.

FIG. 14 is a front view that shows an example of a vector potential coil with respect to a vector potential coil device 1 according to an eleventh embodiment of the present invention.

FIG. 15 is a top view that shows an example of the vector potential coil with respect to the vector potential coil device 1 according to the eleventh embodiment of the present invention.

FIG. 16 is a side view that shows an example of the vector potential coil with respect to the vector potential coil device 1 according to the eleventh embodiment of the present invention.

FIG. 17 is a diagram that shows an example of a support part 10, a vector potential coil 21, and a fixed part 200 of a vector potential coil device 1 according to a twelfth embodiment.

### DETAILED DESCRIPTION

Embodiments of the present invention will be explained below with reference to the drawings.

### First Embodiment

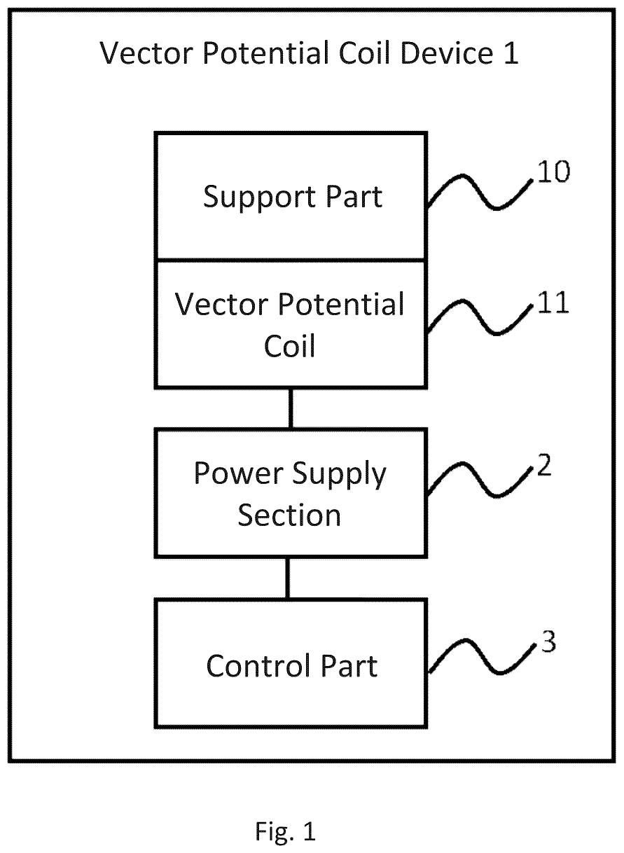

FIG. 1 is a block diagram that shows a configuration of a vector potential coil device 1 for applying an electrical stimulation to skin of a living body according to an embodiment of the present invention.

The vector potential coil device 1 shown in FIG. 1 has a support part (a support) 10, a vector potential coil 11, a power supply section (a power supply) 2, and a control part (a controller) 3.

The support part 10 is a member having a housing (accommodation) space for accommodating a part of the skin of the living body, and has an irradiation surface facing the skin of the living body. In a first embodiment, the support part 10 is a pipe-shaped member. However, the support part 10 may also be, for instance, a plate-shaped member, an arc-shaped member, or a hemispherical member. Further, the above-mentioned living body may be a human or an animal.

The support part 10 may be partially made of a light-transmitting member (material) (for instance, a transparent resin member (material)). In this way, a phototherapy element can be further mounted on the vector potential coil device 1, and electrical and optical stimulations can be applied to the skin at the same time.

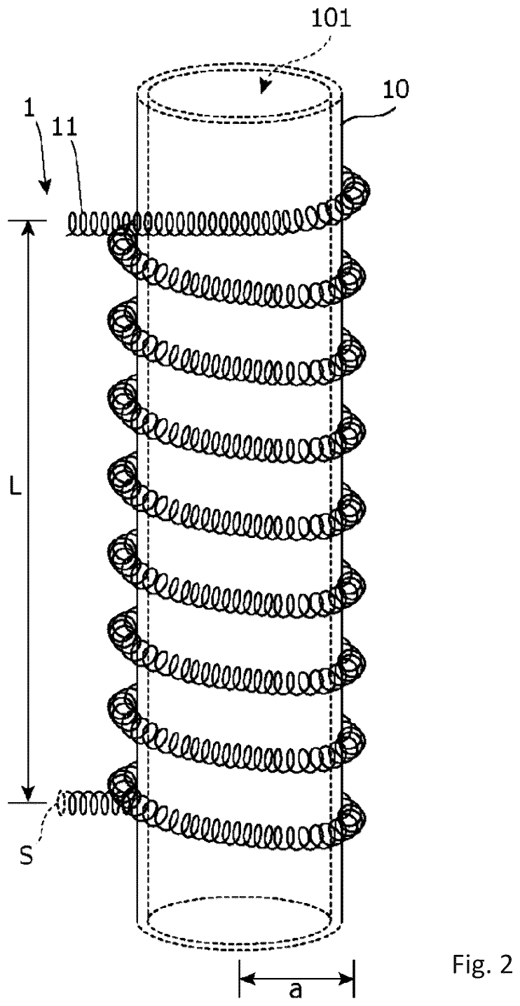

FIG. 2 is a diagram that shows an example of the support part 10 and the vector potential coil device 1 according to the first embodiment.

As shown in, for instance, FIG. 2, the vector potential coil device 1 has a vector potential coil (also referred to as “a VP coil” below) 11 that is arranged in at least one (here, the outside) of a housing space 101 and the outside of the support part 10. In the first embodiment, as shown in, for instance, FIG. 2, the VP coil 11 is a solenoid coil that circulates around a helical (spiral) coil axis. Here, the support part 10 is a cylindrical member. Further, the VP coil 11 has the helical (spiral) coil axis that circulates in a circular shape.

Further, the power supply section 2 generates an alternating current (AC current) based on power from, for instance, a commercial power supply or a battery (a primary battery or a secondary battery), conducts the AC current through the VP coil 11, generates a vector potential that temporally changes (varies in time) in response to the AC current in the above-mentioned housing space 101, and applies an electric field generated based on the vector potential to the above-mentioned skin, thereby applying an electrical stimulation to the above-mentioned skin of the living body. Specifically, when the above-mentioned skin of the living body has the good (higher) conductivity, an AC electric field is applied to the above-mentioned skin, and at the same time, an AC current corresponding to the voltage caused by the electric field is conducted well through the above-mentioned skin. As a result, both the electrical stimulation by the electric field and the electrical stimulation by the current can be applied to the above-mentioned skin. On the other hand, when the above-mentioned skin has the lower conductivity, the AC electric field is applied to the above-mentioned skin of the living body. However, even when the above-mentioned AC current is conducted, it is low. Thus, the electric field stimulation is mainly applied to the above-mentioned skin of the living body. Therefore, when necessary, a highly conductive medicinal solution is applied to the surface of the skin so as to improve the conductivity, and as a result, both the electrical stimulation due to the electric field and the electrical stimulation due to the current can be simultaneously obtained.

When the AC current that is a sine wave with an amplitude Iₘ is conducted through the VP coil 11, an AC voltage V2 shown in the following equation is generated in the housing space 101 in accordance with a temporal change (varying in time) of the vector potential being generated by the AC current, and the AC current according to the AC voltage is conducted through the skin of the living body in the housing space 101.

$${V}_{2} = {μ}_{0} {nN}_{1} S ω ( \sqrt{{a}^{2} + {L}^{2}} - a ) {I}_{m} cos ( ω t ) \qquad [ Equation 1 ]$$

Here, μ₀ is the vacuum permeability, n is the number of turns per unit length of the solenoid in the VP coil 11, N₁ is the number of turns per unit length of the helical VP coil 11, S is the cross-sectional area of the solenoid in the VP coil 11, @ is the angular frequency of the AC current, a is the cross-sectional radius of the helical VP coil 11, L is the length (a distance between both ends) of the VP coil 11, and t is time.

In addition, the control part 3 is such as a computer that executes a control program and controls the power supply section 2 so as to conduct an AC current of a predetermined frequency through the VP coil 11 at a predetermined timing (usually, such as at a predetermined time interval). For instance, the control part 3 may make the power supply section 2 conduct a pulsed AC current with a high-frequency for a short period of time.

Next, an operation of the above-mentioned vector potential coil device will be explained.

The control part 3 controls the power supply section 2, while receiving a signal from a sensor and a clocking device (not shown), and makes the power supply section 2 supply an AC current to the VP coil 11.

At this time, the power supply section 2 generates the AC current with a predetermined waveform (an amplitude and a frequency) at a predetermined timing, and supplies it to the VP coil 11.

A magnetic field is generated along the coil axis by the current flowing through the VP coil 11, and the vector potential is generated in parallel to the current. An intensity of the vector potential in an inner side (an inner direction) of the curvature of the VP coil 11 (i.e., the housing space 101) becomes greater than the vector potential in an outer side (an outer direction) of the curvature of the VP coil 11.

In addition, in the housing space 101, since the vector potential alternates, the AC voltage according to the temporal change (varying in time) is generated as mentioned above. Because the AC current according to the AC voltage is conducted to the skin of the living body in the housing space 101, the electrical stimulation is applied to the skin in the housing space 101. As a result, the treatments of the skin disease or the cosmetic treatment in the housing space 101 are promoted.

After a predetermined time has elapsed, the control part 3 sends a signal to the power supply section 2 to stop the AC current, thereby the current to the VP coil 11 is stopped.

As mentioned above, according to the embodiment, the support part 10 has the housing space 101 for accommodating the skin of the living body. The VP coil 11 is arranged at least one of the housing space 101 and the outside of the support part 10. The power supply section 2 conducts the AC current through the VP coil 11, generates the vector potential corresponding to the AC current in the housing space 101, and applies the electric field generated based on the vector potential to the skin of the living body, thereby applying the electrical stimulation to the skin.

As a result, the electrical stimulation can be applied to the skin without contacting an electrode with the skin of the living body. Therefore, while the occurrence of problems caused by the electrode being used for the electrical stimulation to the skin of the living body is suppressed, the treatments of the skin disease or the cosmetic treatment through the electrical stimulation are promoted.

Further, in a case in which a liquid such as a topical therapeutic agent (therapeutic agent for application) or a beauty serum (beauty liquid) is used, after the agent or the serum (liquid) is applied to the surface of the skin, the vector potential coil device 1 of the present invention is attached. As a result, ingredients of the agent or the beauty serum are ionized and can penetrate more efficiently into the inside of the skin.

### Second Embodiment

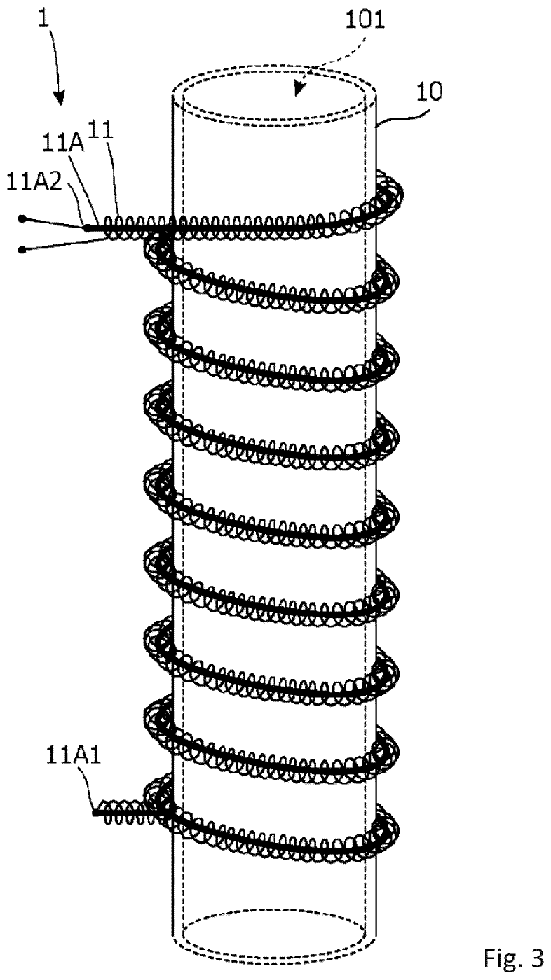

FIG. 3 is a diagram that shows an example of a vector potential coil device 1 according to a second embodiment.

In the second embodiment, as shown in, for instance, FIG. 3, the vector potential coil device 1 further includes, in addition to the VP coil 11, a ferromagnetic member 11A extending along the coil axis within the solenoid coil that configures the VP coil 11.

The ferromagnetic member 11A is formed with a conductive material such as permalloy. Further, one end of the VP coil 11 and one end 11A1 (a first connection point) of the ferromagnetic member 11A are electrically connected to each other, and the power supply section 2 conducts an AC current through the VP coil 11 by applying a voltage to the other end of the VP coil 11 and the other end 11A2 (a second connection point) of the ferromagnetic member 11A.

According to the above configuration, the ferromagnetic member 11A becomes a path for the above-mentioned AC current, and two terminals are arranged at either one of the end sides the VP coil 11, so that wiring from the power supply section 2 to the VP coil 11 and the ferromagnetic member 11A can be easily laid, and at the same time, an area being surrounded by the path through the wiring is relatively narrow. As a result, an unnecessary magnetic field being generated due to the current flowing through the wiring can be suppressed.

Note that the other configurations and operations of the vector potential coil device 1 according to the second embodiment are the same as those explained in any of the other embodiments. Therefore, the explanations of them will be omitted.

### Third Embodiment

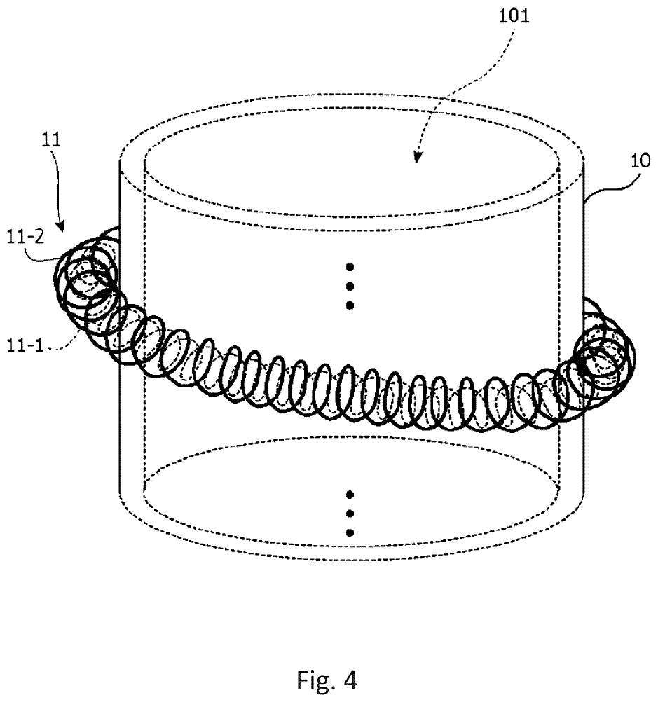

FIG. 4 is a diagram that shows an example of a vector potential coil device 1 (a part) according to a third embodiment.

In the third embodiment, as shown in, for instance, FIG. 4, the VP coil 11 has an inner solenoid coil 11-1 and an outer solenoid coil 11-2 that respectively extend along the same coil axis and that have coil diameters different from each other. Further, one end of the inner solenoid coil 11-1 and one end of the outer solenoid coil 11-2 are electrically connected to each other. Each of the inner solenoid coil 11-1 and the outer solenoid coil 11-2 functions as one VP coil.

Therefore, the VP coil 11 according to the third embodiment electrically has a configuration in which two VP coils are connected in series and in the same phase. In addition, the power supply section 2 conducts the AC current through the vector potential coil by applying the voltage to the other end of the inner solenoid coil 11-1 and the other end of the outer solenoid coil 11-2. As a result, a vector potential due to the inner solenoid coil 11-1 and a vector potential due to the outer solenoid coil 11-2 are generated in the same direction.

Note that the other configurations and operations of the vector potential coil device according to the third embodiment are the same as those explained in any of the other embodiments. Therefore, the explanations of them will be omitted.

### Fourth Embodiment

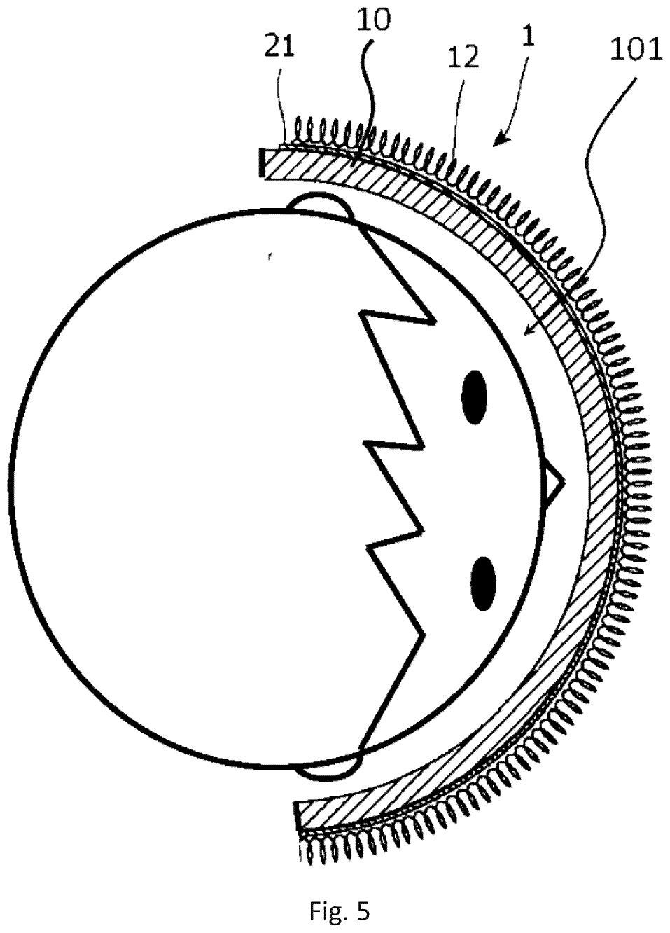

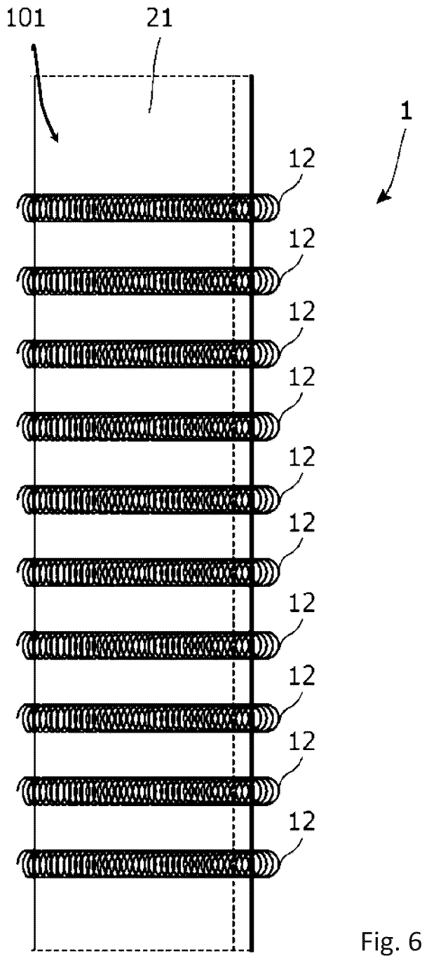

FIG. 5 is a top view that shows an example of a vector potential coil device 1 according to a fourth embodiment. FIG. 6 is a side view that shows an example of the vector potential coil device 1 according to the fourth embodiment.

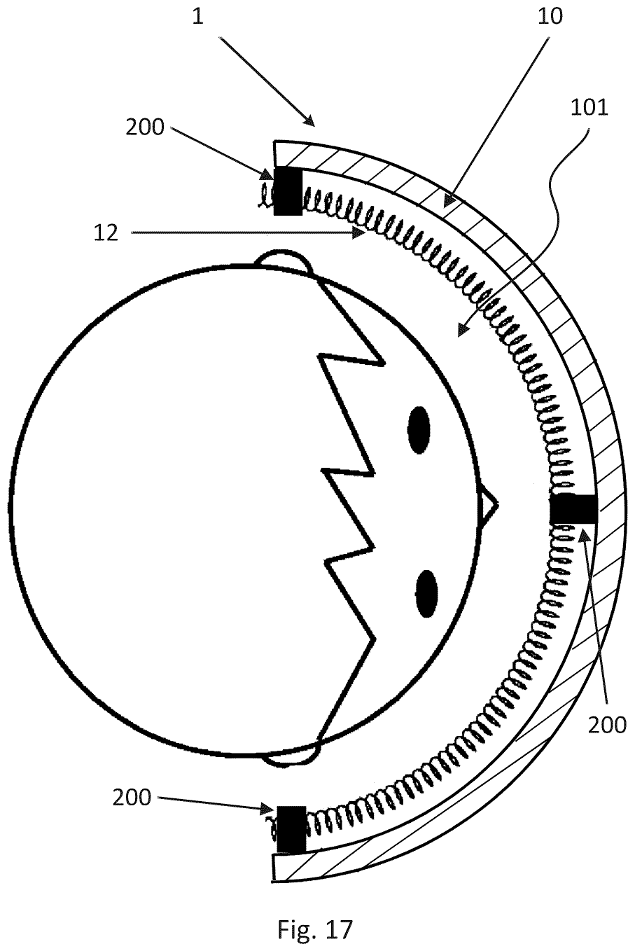

In the fourth embodiment, as shown in, for instance, FIGS. 5 and 6, VP coils 12 are arranged at the housing space 101 (see FIG. 17) and the outside of the support part 10. Specifically, the VP coils 12 are arranged at an outside part 21 of the support part 10 of the housing space 101. Further, the outside part 21 of the support part 10 may be part of an outer surface of the support part. In addition, the outside part 21 of the support part 10 may be a film or a material, such as an insulating film, an insulating material, an adhesive film, and an adhesive.

In addition, in the fourth embodiment, the VP coil 12 is a solenoid coil extending along a curved coil axis and has openings in the circumferential direction. In other words, the coil axis of the VP coil 12 does not go around once (one revolution) or more.

For instance, the above-mentioned coil axis of the VP coil 12 is in a circular arc shape, and an angle (central angle) from one end to the other end of the VP coil 12 (the coil axis of the VP coil) when viewed from a center (here, the central axis of the housing space 101) of a circle including the coil axis (i.e., the circular arc) is less than 360 degrees. As a result, the above-mentioned opening is formed. For instance, the central angle may be 180 degrees or may be less than 180 degrees. However, the larger the central angle is, the greater the intensity of the vector potential in the inner side of the curvature becomes. Thus, it is preferred that the central angle is large. The central angle is any angle greater than 0 degrees and less than 360 degrees, and may further be (a) any angle greater than 0 degrees and equal to or less than 180 degrees, (b) any angle greater than 0 degrees and equal to or less than 90 degrees, (c) any angle greater than 0 degrees and equal to or less than 45 degrees, or (d) any angle equal to or greater than 0.5 degrees and less than 360 degrees, and further, (e) any angle equal to or greater than 0.5 degrees and equal to or less than 180 degrees, (f) any angle equal to or greater than 0.5 degrees and equal to or less than 90 degrees, (g) any angle equal to or greater than 0.5 degrees and equal to or less than 45 degrees, (h) any angle equal to or greater than 0.5 degrees and equal to or less than 25 degrees, or (i) any angle equal to or greater than 2 degrees and less than 360 degrees, further, (j) any angle equal to or greater than 2 degrees and equal to or less than 180 degrees, (k) any angle equal to or greater than 2 degrees and equal to or less than 90 degrees, (1) any angle equal to or greater than 2 degrees and equal to or less than 45 degrees, (m) any angle equal to or greater than 2 degrees and equal to or less than 25 degrees, or (n) any angle equal to or greater than 5 degrees and less than 360 degrees, further, (o) any angle equal to or greater than 5 degrees and equal to or less than 180 degrees, (p) any angle equal to or greater than 5 degrees and equal to or less than 90 degrees, (q) any angle equal to or greater than 5 degrees and equal to or less than 45 degrees, or (r) any angle equal to or greater than 5 degrees and equal to or less than 25 degrees.

A vector potential due to a current that is conducted through the VP coil 12 is weakened as it moves away from the current. However, since the VP coil 12 (its coil axis) is curved as mentioned above, an intensity of the vector potential becomes greater at an inner side (an inner direction) of the curvature (a curvature center in the case of a circular arc shape). Specifically, the vector potentials being generated by the current at each position of the VP coil 12 overlap at the inner side of the curvature.

In addition, the vector potential coil device according to the fourth embodiment has a plurality of VP coils 12 that are identical to one another. The VP coils 12 are arranged along an axial direction of the pipe-shaped support part 10.

The power supply section 2 conducts an AC current through the plurality of VP coils 12, generates a vector potential corresponding to the AC current in the housing space 101, and conducts an AC current corresponding to a voltage generated based on the vector potential to a liquid in the housing space 101, thereby applying an electrical stimulation to the skin of the living body. The VP coils 12 are electrically connected to one another in series or in parallel and generate the vector potential in the same direction at each point in time.

Note that the other configurations and operations of the vector potential coil device according to the fourth embodiment are the same as those explained in any of the other embodiments. Therefore, the explanations of them will be omitted.

### Fifth Embodiment

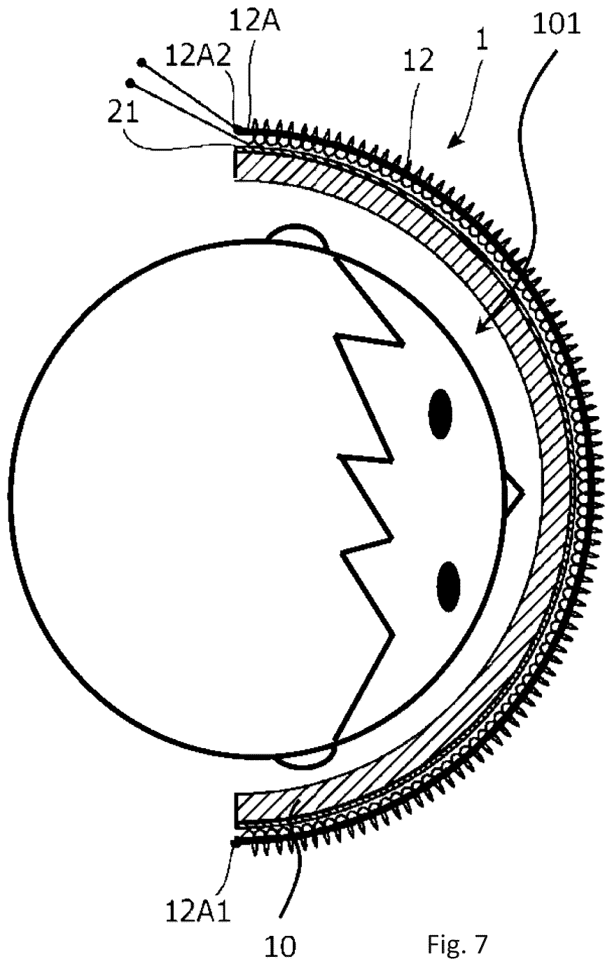

FIG. 7 is a top view that shows an example of a vector potential coil device 1 according to a fifth embodiment.

In the fifth embodiment, as shown in, for instance, FIG. 7, the vector potential coil device 1 further has a ferromagnetic member 12A that extends along the coil axis within the solenoid coil serving as the VP coil 12. The ferromagnetic member 12A is formed with a conductive material such as permalloy. Further, one end of the VP coil 12 and one end 12A1 (a first connection point) of the ferromagnetic member 12A are electrically connected to each other. The power supply section 2 conducts an AC current through the VP coil 12 by applying a voltage to the other end of the VP coil 12 and the other end 12A2 (a second connection point) of the ferromagnetic member 12A.

Since the vector potential is enhanced according to an effective magnetic permeability of the ferromagnetic member 12A, the intensity of the vector potential becomes greater at the inner side of the curvature (a curvature center in the case of a circular arc shape).

Since the coil axis of the VP coil 12 does not go around once (one revolution) or more, the distance between both ends of the VP coil 12 is large. However, the ferromagnetic member 12A becomes a path for the current and two of the terminals are arranged on either end side of the VP coil 12, so that the wiring from the power supply section 2 to the VP coil 12 and the ferromagnetic member 12A can be easily laid, and at the same time, the area being encircled by the path through the wiring is relatively narrow. As a result, an unnecessary magnetic field being generated due to the current flowing through the wiring can be suppressed.

Note that the other configurations and operations of the vector potential coil device according to the fifth embodiment are the same as those explained in the fourth embodiment. Therefore, the explanations of them will be omitted.

### Sixth Embodiment

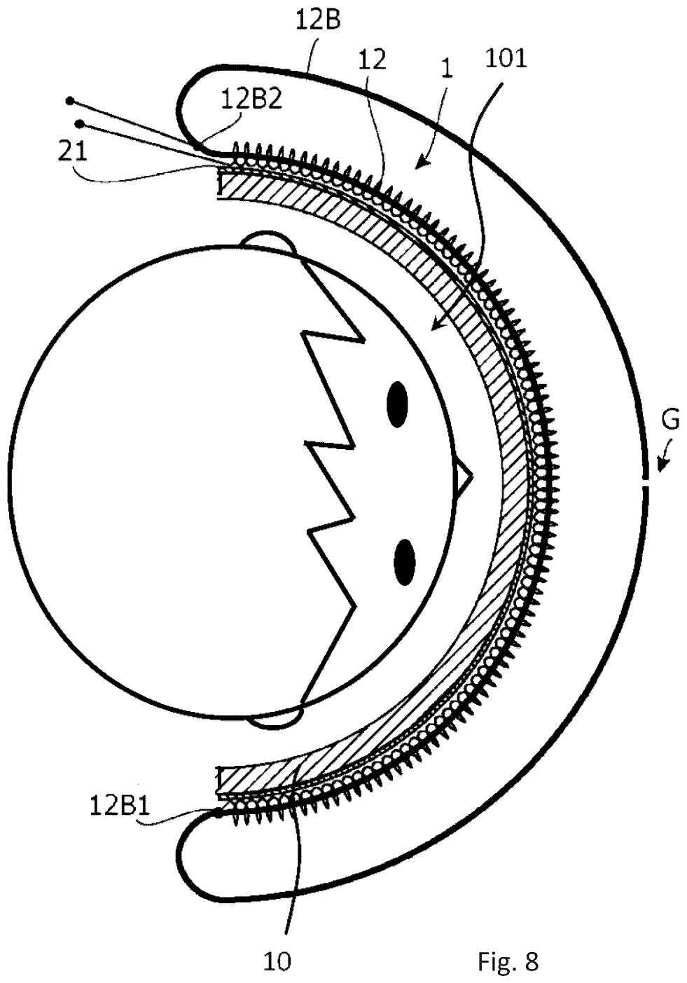

FIG. 8 is a top view that shows an example of a vector potential coil device 1 according to a sixth embodiment.

In the sixth embodiment, as shown in, for instance, FIG. 8, the vector potential coil device 1 further has a ferromagnetic member 12B that extends along the coil axis within the solenoid coil serving as the VP coil 12. The ferromagnetic member 12B is conductive, and one end of the VP coil 12 and a connection point 12B1 (a point on one end side of the VP coil 12, a first connection point) of the ferromagnetic member 12B are electrically connected to each other. The power supply section 2 conducts an AC current through the VP coil 12 by applying a voltage to the other end of the VP coil 12 and a connection point 12B2 (a point on the other end side of the VP coil 12, a second connection point) of the ferromagnetic member 12B.

Furthermore, the ferromagnetic member 12B is curved toward an outside (in the outer side (direction) of the curvature) from the connection points 12B1 and 12B2, and the ferromagnetic member 12B forms a closed magnetic path via a gap G. Note that the gap G prevents the current from being conducted through an outside part of the curvature of the VP coil 12.

Further, it is preferred that transition portions between the inner portion and the outer portion of the ferromagnetic member 12B are made into a continuous and smooth curve shape without any steep bend portions, in order to reduce a leakage of a magnetic flux and reduce the influence of the decrease of the magnetic permeability due to bending processing. Moreover, the ferromagnetic member 12B may be formed by connecting a plurality of members.

Note that the other configurations and operations of the vector potential coil device according to the sixth embodiment are the same as those explained in the fourth embodiment. Therefore, the explanations of them will be omitted.

### Seventh Embodiment

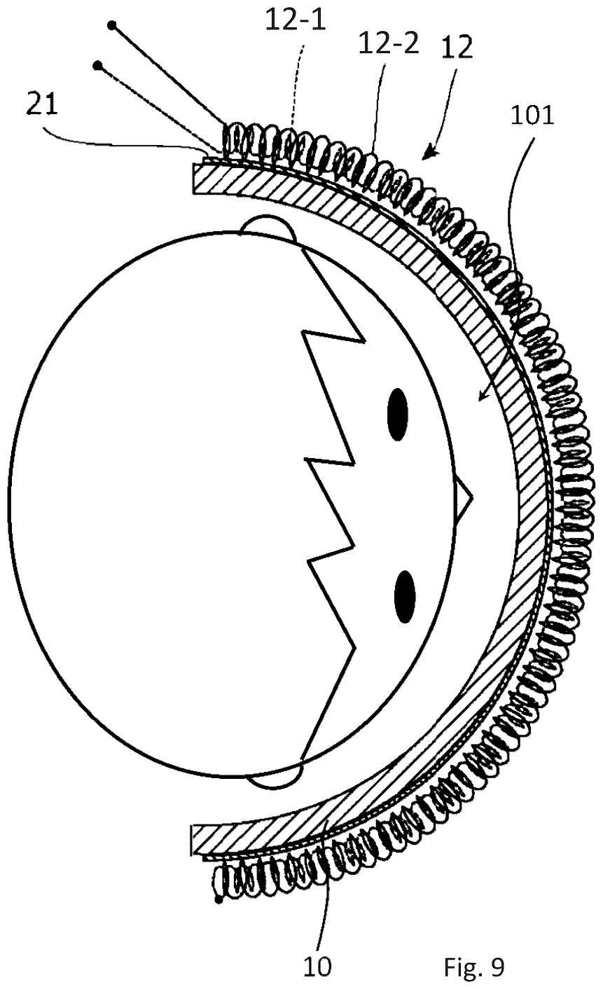

FIG. 9 is a top view that shows an example of a vector potential coil device 1 according to a seventh embodiment.

In the seventh embodiment, as shown in, for instance, FIG. 9, the VP coil 12 has an inner solenoid coil 12-1 and an outer solenoid coil 12-2 that respectively extend along the same coil axis and in which coil diameters are different from each other. One end of the inner solenoid coil 12-1 and one end of the outer solenoid coil 12-2 are electrically connected to each other. The power supply section 2 conducts the AC current through the VP coil 12 by applying a voltage to the other end of the inner solenoid coil 12-1 and the other end of the outer solenoid coil 12-2.

Each of the inner solenoid coil 12-1 and the outer solenoid coil 12-2 functions as one VP coil. Therefore, the VP coil 12 according to the seventh embodiment electrically has a configuration in which two VP coils are connected in series and in the same phase. In addition, the power supply section 2 conducts the AC current through the vector potential coil by applying the voltage to the other end of the inner solenoid coil 12-1 and the other end of the outer solenoid coil 12-2. As a result, a vector potential due to the inner solenoid coil 12-1 and a vector potential due to the outer solenoid coil 12-2 are generated in the same direction.

Note that the other configurations and operations of the vector potential coil device according to the seventh embodiment are the same as those explained in the fourth embodiment. Therefore, the explanations of them will be omitted.

### Eighth Embodiment

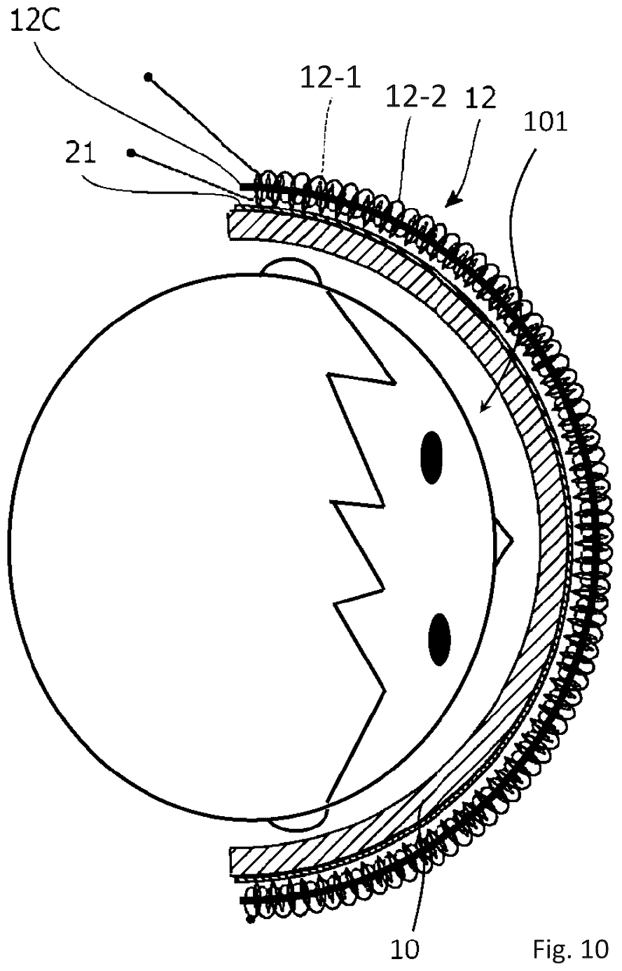

FIG. 10 is a top view that shows an example of a vector potential coil device 1 according to an eighth embodiment.

In the eighth embodiment, as shown in, for instance, FIG. 10, the vector potential coil device 1 further has a ferromagnetic member 12C that extends along the coil axis of the inner solenoid coil 12-1 and the outer solenoid coil 12-2 serving as the VP coil 12. Further, the ferromagnetic member 12C is not electrically connected to the inner solenoid coil 12-1 and the outer solenoid coil 12-2.

Note that the other configurations and operations of the vector potential coil device according to the eighth embodiment are the same as those explained in the seventh embodiment. Therefore, the explanations of them will be omitted.

### Ninth Embodiment

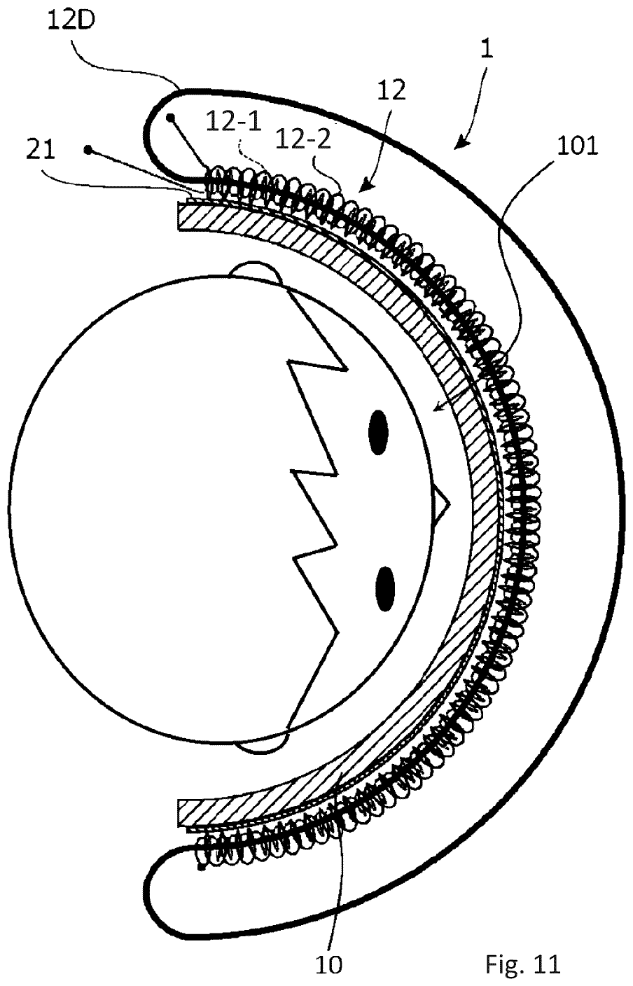

FIG. 11 is a top view that shows an example of a vector potential coil device 1 according to a ninth embodiment.

In the ninth embodiment, as shown in, for instance, FIG. 11, the vector potential coil device 1 has a ferromagnetic member 12D that extends along the coil axis of the inner solenoid coil 12-1 and the outer solenoid coil 12-2 serving as the VP coil 12.

Furthermore, the ferromagnetic member 12D extends toward an outside from both ends of the VP coil 12 while curving and forms a closed magnetic path. Further, the ferromagnetic member 12D is not electrically connected to the inner solenoid coil 12-1 and the outer solenoid coil 12-2. The ferromagnetic member 12D may not have to be conductive. In addition, a gap is not provided in the ferromagnetic member 12D. In the ninth embodiment, an AC current is conducted through the inner solenoid coil 12-1 and the outer solenoid coil 12-2 but is not conducted through the ferromagnetic member 12D. Therefore, the ferromagnetic member 12D may not require conductivity or the gap.

Note that the other configurations and operations of the vector potential coil device according to the ninth embodiment are the same as those explained in the seventh embodiment. Therefore, the explanations of them will be omitted.

### Tenth Embodiment

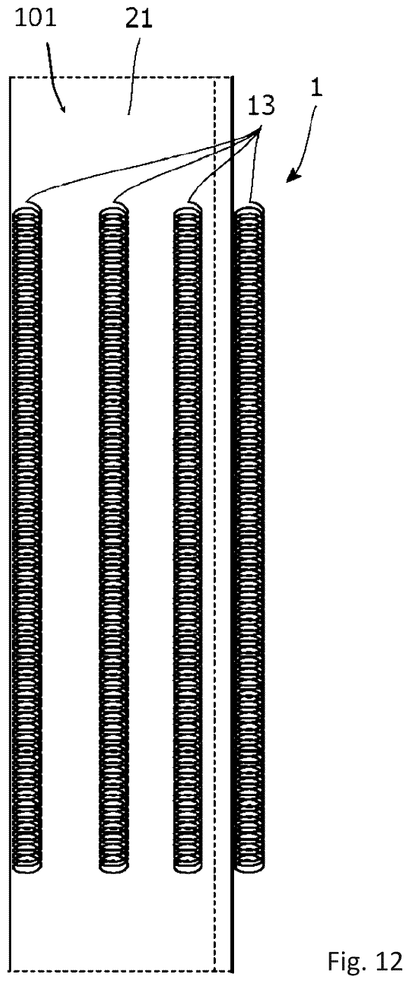

FIG. 12 is a side view that shows an example of a vector potential coil device 1 according to a tenth embodiment. FIG. 12 is a top view that shows an example of the vector potential coil device 1 according to the tenth embodiment.

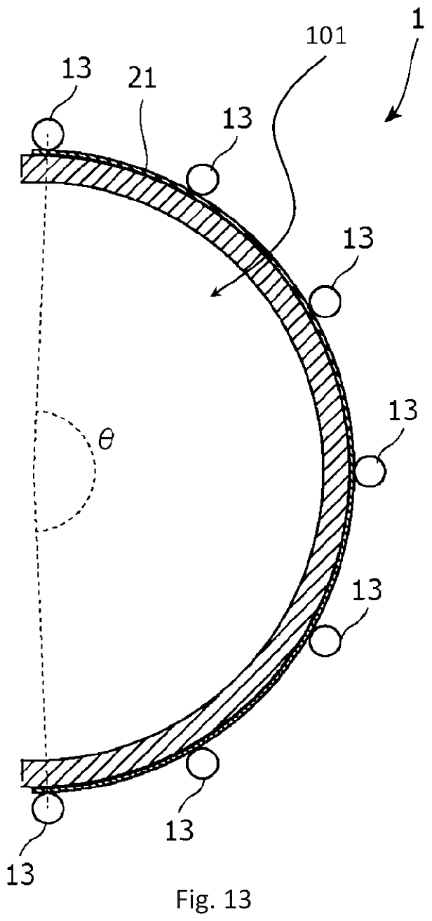

As shown in, for instance, FIGS. 12 and 13, the vector potential coil device according to the tenth embodiment has a plurality of VP coils 13 that are identical to one another. In the tenth embodiment, the VP coils 13 are solenoid coils, respectively, that are identical to one another and that respectively has a linear coil axis. These VP coils 13 are arranged along the circumferential direction of the pipe-shaped support part 10.

The power supply section 2 conducts an AC current through the plurality of VP coils 13, generates a vector potential corresponding to the AC current in the housing space 101, and conducts an AC current corresponding to a voltage generated based on the vector potential to a liquid in the housing space 101, thereby applying an electrical stimulation to the skin of the living body. The VP coils 13 are electrically connected to one another in series or in parallel and generate the vector potential in the same direction at each point in time.

The plurality of VP coils 13 are arranged within an angular range of a predetermined central angle θ (here, at an interval of an equal angle) from the center of the housing space 101. Since the vector potentials of two of the VP coils 13 are canceled out at the intermediate position between the two VP coils 13, for instance, the central angle θ is set to be any angle less than 180 degrees.

Note that the other configurations and operations of the vector potential coil device according to the tenth embodiment are the same as those explained in any of the other embodiments. Therefore, the explanations of them will be omitted.

### Eleventh Embodiment

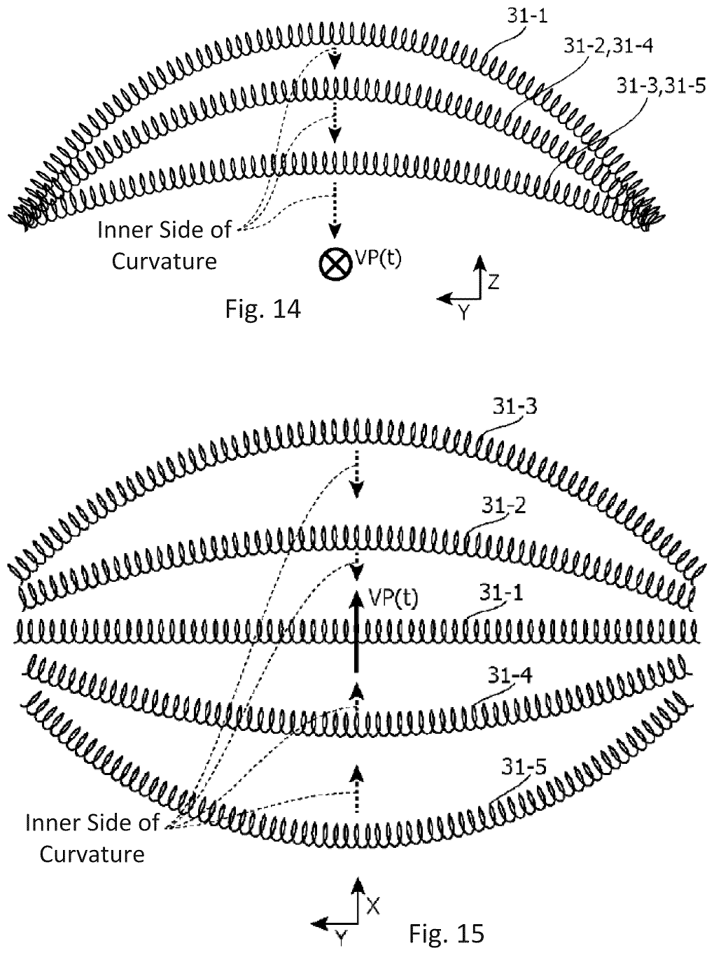

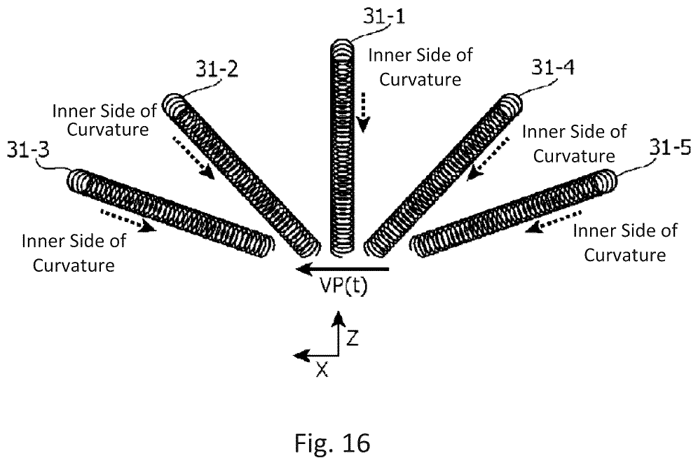

FIG. 14 is a front view that shows an example of vector potential coils with respect to a vector potential coil device 1 according to an eleventh embodiment of the present invention. FIG. 15 is a top view that shows an example of the vector potential coils with respect to the vector potential coil device 1 according to the eleventh embodiment of the present invention. FIG. 16 is a side view that shows an example of the vector potential coils with respect to the vector potential coil device 1 according to the eleventh embodiment of the present invention.

The vector potential coil device 1 according to the eleventh embodiment has a plurality of vector potential coils 31-1-31-5. As shown in, for instance, FIGS. 14-16, the plurality of vector potential coils 31-1-31-5 are respectively wound along (around) a curved coil axis, and are arranged so that the inner sides (directions) of the curvatures of the coil axes (in other words, planes that include the coil axes) cross mutually. As shown in, for instance, FIG. 16, the plurality of vector potential coils 31-1-31-5 are arranged so that the planes that include the coil axes of the plurality of vector potential coils 31-1 to 31-5 are in parallel to the Y-axis direction, and at the same time, the angle intervals of the inclination angles of these planes with respect to the X-axis direction are substantially the same. In addition, here, the inclination angle of the vector potential coil 31-1 is 90 degrees.

Note that, here, the vector potential coil device 1 has five of the vector potential coils 31-1-31-5. However, the vector potential coil device 1 may have the vector potential coils 31-1-31-M in the same manner as the configuration described above. The number M is either 2-4 coils or 6 or more coils.

For instance, the shape (such as the curvature) and the arrangement of the coil axes are determined so that the coil axes of the plurality of vector potential coils 31-1-31-5 are included in a single partial spherical surface (for instance, a semispherical surface). Further, the application target is arranged at the center of the spherical surface that includes that partial spherical surface (in other words, the center of curvatures of all of the coil axes). Further, the shape (such as the curvature) and the arrangement of the coil axes may be determined so that the coil axes of the plurality of vector potential coils 31-1-31-5 are included in a curved surface (a partial aspherical surface) other than a single partial spherical surface.

Further, the plurality of vector potential coils 31-1-31-5 respectively generate a vector potential according to the AC current in the same manner as the above-mentioned embodiments. The vector potentials by the plurality of vector potential coils 31-1-31-5 are synthesized so that a vector potential VP (t) is obtained. Here, the power supply section 2 conducts the AC current through the plurality of vector potential coils 31-1-31-5 so that the amplitude of the synthesized vector potential VP (t) becomes maximum (for instance, in the same phase mutually).

Note that the other configurations and operations of the vector potential coil device 1 according to the eleventh embodiment are the same as those explained in any of the other embodiments. Therefore, the explanations of them will be omitted. In other words, any of the above-mentioned ferromagnetic members may be respectively arranged along the coil axes of the plurality of vector potential coils 31-1-31-5.

As mentioned above, according to the vector potential coil device 1 according to the above-mentioned eleventh embodiment, it is possible to concentrate the vector potentials in the inner sides (direction) of the curvatures of the plurality of vector potential coils 31-1-31-5 and apply a high-intensity vector potential to the application target.

### Twelfth Embodiment

FIG. 17 is a diagram that shows an example of a support part 10, a vector potential coil 21, and fixed parts 200 of a vector potential coil device 1 according to a twelfth embodiment.

In the twelfth embodiment, as shown in, for instance, FIG. 17, the VP coil 12 is arranged at an inside of a support part 10 in a housing space 101. The VP coil 12 is fixed to the inner side of the support part 10 at three locations via the fixed parts 200.

Note that the other configurations and operations of the vector potential coil device 1 according to the twelfth embodiment are the same as those explained in any of the other embodiments. Therefore, the explanations of them will be omitted.

In addition, the other features shown in the other embodiments can be combined with or added to the configuration shown in FIG. 17. For example, the VP coil 12 is a solenoid coil extending along a curved coil axis and has openings in the circumferential direction. In other words, the coil axis of the VP coil 12 does not go around once (one revolution) or more.

Note that various changes and modifications to the embodiments described above will be apparent to one having ordinally skill in the art. Such the changes and modifications may be made without departing from the spirit and scope of the subject matter and without diminishing the intended advantages. That is, it is intended that such the changes and modifications are included within the scope of the claims.

For instance, in the above-mentioned third and seventh to ninth embodiments, the VP coil 12 has the two-layer structure, which has the inner solenoid coil 12-1 and the outer solenoid coil 12-2, in the radial direction. However, the number of layers may be four or more as long as the number of layers is an even number. In this case, either one of the ends of the solenoid coil 12-i is connected to the solenoid coil 12-(i+1) in the next layer so that the solenoid coils 12-i in all layers are electrically connected in series.

In addition, in the above-mentioned third to eleventh embodiments, the VP coils 11, 12, 13, or 31-1 to 31-5 are arranged at only one side of the skin of the living body. However, the VP coils 11 may be arranged on both or more sides with respect to the outside of the living body. Further, since the electric field intensity in the housing space 101 of the VP coils 11, 12, 13, or 31-1 to 31-5 varies depending on the location, when necessary, the skin of the living body can be placed in a different location in the housing space.

### INDUSTRIAL APPLICABILITY

The present invention can be applicable to, for instance, the treatments of the skin diseases or the cosmetic treatments on the living body. In particular, it is expected to be used to treat acne, hyperhidrosis (excess sweating), burns, and a sutured area of skin of post-surgery (sutures on a surgically treated skin).

What is claimed is:

1. A vector potential coil device comprising:
a support part having a housing space that accommodates skin of a living body;
a vector potential coil arranged at at least one of the housing space and an outside of the support part; and
a power supply section that conducts an alternating current through the vector potential coil, generates a vector potential corresponding to the alternating current in the housing space, and provides an electrical stimulation to the skin by applying an electric field generated based on the vector potential to the skin of the living body.

2. The vector potential coil device according to claim 1,
wherein the vector potential coil is a solenoid coil that circulates around a helical coil axis.

3. The vector potential coil device according to claim 1,
wherein the vector potential coil is a solenoid coil that extends along a curved coil axis and has an opening in a circumferential direction.

4. The vector potential coil device according to claim 2, further comprising:
a ferromagnetic member extending along the coil axis within the solenoid coil,
wherein the ferromagnetic member has conductivity,
one end of the vector potential coil and a first connection point of the ferromagnetic member are electrically connected to each other, and
the power supply section applies a voltage to the other end of the vector potential coil and a second connection point of the ferromagnetic member to conduct a current through the vector potential coil.

5. The vector potential coil device according to claim 2,
wherein the vector potential coil includes an inner solenoid coil and an outer solenoid coil each extending along the same coil axis,
one end of the inner solenoid coil and one end of the outer solenoid coil are electrically connected to each other, and
the power supply section applies a voltage to the other end of the inner solenoid coil and the other end of the outer solenoid coil to conduct a current through the vector potential coil.

6. The vector potential coil device according to claim 1, further comprising:
a plurality of vector potential coils including the vector potential coil,
wherein the plurality of vector potential coils are arranged along an axial direction or a circumferential direction of the support part, and
the power supply section conducts an alternating current through the plurality of vector potential coils, generates a vector potential corresponding to the alternating current in the housing space, and
provides an electrical stimulation to the skin by applying an electric field generated based on the vector potential to the skin of the living body.
